(19) United States
(12) Patent Application Publication
(10) Pub. No.: US 2006/0167354 A1
(43) Pub. Date: Jul. 27, 2006

(54) System for fixing a biomedical electrode

(76) Inventors: Pierre-Yves Muller, Collonge-Bellerive (CH); Eric Padoy, Biere (CH)

Correspondence Address: James H Patterson;Patterson Thuente Skaar & Christensen, 4800 IDS Center, 80 South 8th Street, Minneapolis, MN, 55402, US

(21) Appl. No.: 10/525,309

(22) PCT Filed: Jul. 22, 2003
(86) PCT No.: PCT/CH03/00493

§ 371 Date: Feb. 22, 2005

(30) Foreign Application Priority Data
Aug. 22, 2002 (CH) 1440/02

(51) Int. Cl. A61B 5/04

(52) U.S. Cl. 600/394

(57) ABSTRACT

The invention relates to an assembly comprising a biomedical electrode, a cable and elastic snap-in fixing means which are intended to fix the electrode to the cable. The aforementioned fixing means comprise a male element and a female element. The invention is characterized in that the male element comprises a hole and in that the female element comprises a projection which is shaped such that it can be housed in said hole.

The present invention relates to a system for fastening a biomedical electrode. More precisely, it relates to a system for fastening a biomedical electrode that includes resilient snap-on fastening means.

The patent documents U.S. Pat. No. 3,882,853, U.S. Pat. No. 4,660,562, CH 668 690, U.S. Pat. No. 6,276,054, FR 1 314 201 and WO 01/17423 disclose fastening systems for an electrode to be used only once. Usually, the electrode consists of a flexible element of plane shape that includes an adhesive face, intended for fastening the electrode to a person's skin, and a face provided with a male conductive element. Each electrode is associated with a cable provided at one of its ends with a female element intended to accommodate the aforementioned male element.

The object of the present invention is to improve the connection between the male element and the female element, in particular so as to obtain better mechanical strength and better conduction between the electrode and the cable, and also to make it easier to position the female element on the male element when fastening them together.

The invention relates to an assembly comprising a biomedical electrode, a cable and resilient snap-on fastening means intended to fasten said electrode to said cable, said fastening means comprising a male element and a female element, characterized in that the male element has an orifice and in that the female element has a protuberance, the latter being shaped so as to be housed in said orifice when fastening the two elements together.

According to a preferred embodiment of the invention, the male element is placed on the electrode and the female element is situated on one end of the cable.

According to one variant, the fastening system is identical to that of the prior art disclosed above. However, it differs in that the male element includes on its upper face an orifice and in that the female element includes in its cavity a protuberance intended to be housed in the orifice of the male element once the two elements have been fastened together.

To make it easier to fasten the two elements, but also to keep them in place once they have been fastened, it is preferred to choose a fastening system characterized in that the male element comprises a first portion of substantially frustoconical shape, located on the side facing the female element, which first portion is placed on a second portion of substantially frustoconical shape, which is the reverse of that of the first portion. Relative to the principal vertical axis of the male element, the angle made by the side wall of the first portion is less than the angle made by the side wall of the second portion.

According to variant of the invention, the angle made by the side wall of the first portion lies between 50 and 150 and the angle made by the side wall of the second portion lies between 30° and 50°.

One particularly advantageous configuration is obtained when the angle made by the side wall of the first portion is approximately equal to 80 and when the angle made by the side wall of the second portion is approximately equal to 40°.

The invention also relates to an electrode as described above and to a cable provided with a female plug as described above.

One embodiment of the invention will be described in detail below by means of the following figures:

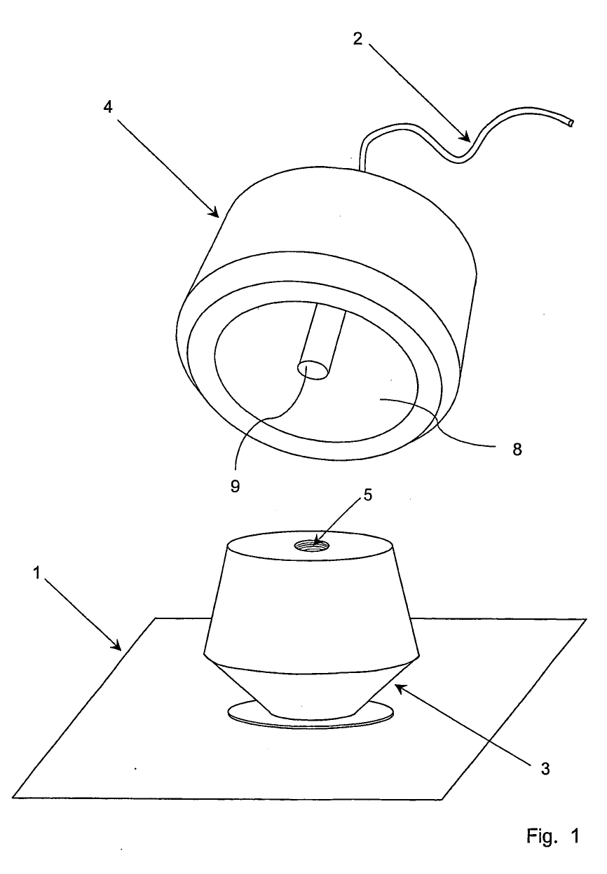

FIG. 1 illustrates an assembly comprising an electrode, a cable and fastening means.

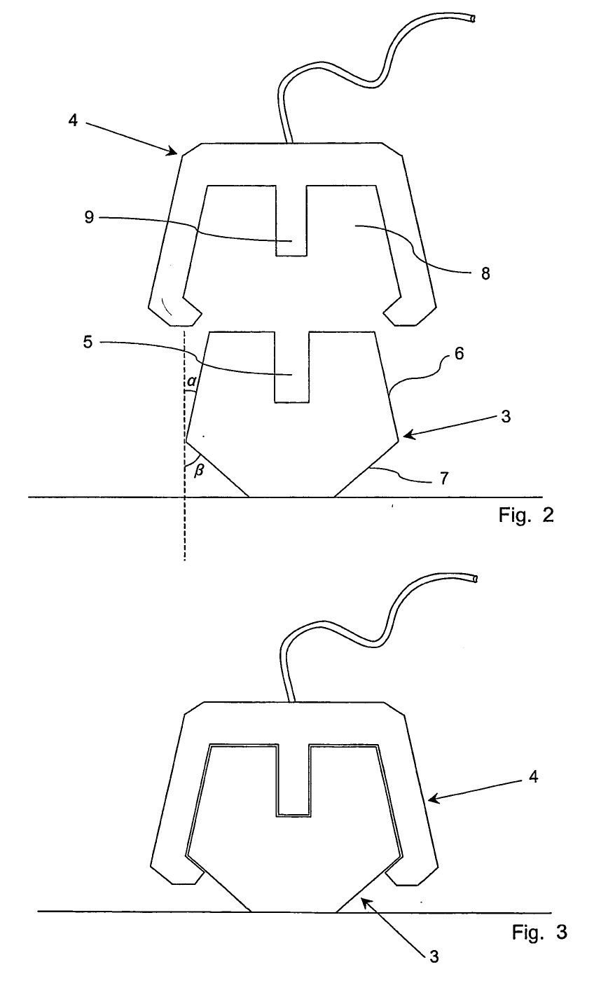

FIG. 2 shows a cross section of the male element and of the female element, the elements being separate from each other.

FIG. 3 shows a cross section of the male element and of the female element, the elements being fastened together.

As may be seen in FIG. 1, a flexible electrode 1 of flat shape is provided on its upper face with a male element 3. The latter (see also FIG. 2) consists mainly of a first part 6 of frustoconical shape and a second part 7, also of frustoconical shape but the reverse of the first part. Placed on the upper face of the male element is an orifice 5.

The female element 4 comprises a cavity 8 intended to surround the male element 3.

The inside of the cavity 8 includes a pin 9, preferably made of a conductive material.

Once the two elements have been fastened together (see FIG. 3), the pin 9 is in intimate contact with the walls of the orifice 5 of the male element 3.

As may be seen in FIG. 2, the angle α defined between the vertical and the side wall of the upper portion 6 of the male element 3 is less than the angle β defined between the vertical and the side wall of the lower portion of the male element 3.

It has been found that an angle α approximately equal to 8° and an angle β approximately equal to 40° constitutes a good compromise, in order to provide easy fastening at the same time as an effective retention force.

It goes without saying that the invention is not limited to one particular shape of electrode. Likewise, the choice of materials and the geometry of the various elements are therefore not limited to what has been described in the example discussed above.

Furthermore, the invention is not limited to one particular field of use. Preferably, it is aimed at the field of muscular electrostimulation, but it may also be applicable elsewhere, for example for establishing an electrocardiogram.

1-8. (canceled)

9. An assembly comprising: a biomedical electrode, a cable, and a resilient snap-on fastening means to fasten said electrode to said cable, said fastening means comprising a male element and a female element, said female element having a cavity adapted to surround said male element, wherein the male element has an orifice and the female element has a protuberance, the protuberance being shaped so as to be housed in said orifice.

10. The assembly as claimed in claim 9, wherein the male element is fastened to the electrode and the female element is fastened to the cable.

11. The assembly as claimed in claim 9, wherein the male element comprises a first portion of substantially frustoconical shape, located on a side facing the female element, which first portion is placed on a second portion of substantially frustoconical shape, which is a reverse of that of the first portion.

12. The assembly as claimed in claim 11, wherein, relative to a principal vertical axis of the male element, an angle made by a side wall of the first portion is less than an angle made by a side wall of the second portion.

13. The assembly as claimed in claim 12, wherein the angle made by the side wall of the first portion lies between 5° and 1520 and the angle made by the side wall of the second portion lies between 30° and 50°.

14. The assembly as claimed in claim 13, wherein the angle made by the side wall of the first portion is approximately equal to 8° and the angle made by the side wall of the second portion is approximately equal to 40°.

15. A biomedical electrode comprising:
a male fastening element comprising an orifice, the male fastening element adapted to be received by a female fastening element comprising a cavity, the orifice adapted to house a protuberance of the female fastening element.

16. The biomedical electrode as claimed in claim 15, wherein the male element comprises a first portion of substantially frustoconical shape, the first portion arranged on a second portion of substantially frustoconical shape a reverse of the first portion.

17. The biomedical electrode as claimed in claim 16, wherein, relative to a principal vertical axis of the male element, an angle made by a side wall of the first portion is less than an angle made by a side wall of the second portion.

18. The biomedical electrode as claimed in claim 17, wherein the angle made by the side wall of the first portion lies between 5° and 15° and the angle made by the side wall of the second portion lies between 30° and 50°.

19. The biomedical electrode as claimed in claim 18, wherein the angle made by the side wall of the first portion is approximately equal to 8° and the angle made by the side wall of the second portion is approximately equal to 40°.

20. A cable for a biomedical electrode comprising:
a resilient female fastening element comprising a cavity and a protuberance, the cavity adapted to surround a male fastening element and the protuberance adapted to be housed in an orifice of the male fastening element.

21. The cable as claimed in claim 20, wherein the cavity comprises a substantially frustoconical shape, the protuberance extending therein, and an inwardly angled integral snap ring portion, the snap ring portion adapted to engage the male fastening element.

22. The cable as claimed in claim 21, wherein, relative to a principal vertical axis of the protuberance, an angle made by an interior side wall of the cavity is less than an angle made by the inwardly angled integral snap ring portion.
